(19) United States
(12) Patent Application Publication
(10) Pub. No.: US 2018/0303674 A1
(43) Pub. Date: Oct. 25, 2018

(54) SKIN-PROTECTIVE HYDROGEL WOUND DRESSING

(71) Applicant: Cell Constructs I, LLC, Marietta, GA (US)

(72) Inventors: Thomas H. Barrows, Austell, GA (US); Alex I. MacDougall, Atlanta, GA (US); Chris A. Ingraham, Marietta, GA (US); Luan K. Vu, Lawrenceville, GA (US)

(21) Appl. No.: 15/959,878

(22) Filed: Apr. 23, 2018

Related U.S. Application Data
(60) Provisional application No. 62/489,074, filed on Apr. 24, 2017.
(60) Provisional application No. 62/512,307, filed on May 30, 2017.

(51) Int. Cl. A61F 13/00; A61L 15/32; A61L 15/28; A61L 15/20; A61L 15/44; A61L 15/42

(52) U.S. Cl. CPC A61F 13/00042; A61F 13/00012; A61F 13/00017; A61F 13/00029; A61F 13/00059; A61F 13/00063; A61F 13/00068; A61F 13/00991; A61L 15/32; A61L 15/28; A61L 15/20; A61L 15/44; A61L 15/425; A61F 13/00995

(57) ABSTRACT

Disclosed herein is a hydrogel wound dressing having a wound-contact side and a non-wound-contact side wherein the rate of moisture evaporation from said non-wound contact side is faster than the rate of moisture evaporation from said wound contact side, thereby causing said dressing to curl toward said non-wound contact side upon drying. Optionally, the hydrogel wound dressing can be comprised of two materials having different rates of moisture evaporation bonded together to form a bilayer structure.

### CROSS-REFERENCE TO RELATED APPLICATIONS

This application claims benefit to U.S. Provisional Application No. 62/489,074, filed Apr. 24, 2017, and U.S. Provisional Application No. 62/512,307, filed May 30, 2017, the entire contents of both of which are incorporated herein by reference.

### TECHNICAL FIELD

The technical field includes wound dressings, and in particular, biomaterials suitable for contact with open wounds that facilitate optimal wound healing.

### BACKGROUND

Most superficial wounds involving broken skin on healthy individuals heal rapidly without the need for intervention beyond keeping the wound protected and free of contamination. However, many conditions such as old age, diabetes, poor circulation of blood in the legs, and pressure ischemia as seen in bed-ridden invalids can delay or even prevent wound healing. Dealing with such chronic wounds continues to be a major unsolved medical problem causing untold human suffering and an enormous cost burden to the healthcare system.

Clinical research and scientific studies utilizing animal models since the 1960s have established that keeping a wound moist rather than dry is the ideal condition for achieving an optimal rate of wound healing. Despite this knowledge, however, a large portion of the wound care market is still served by products that do not contribute to maintaining a moist wound healing environment. The primary reason for lack of adoption of this proven scientific benefit is that moisture is difficult to administer, meter, and confine to the precise location where needed. Additionally, excess moisture is especially harmful to newly healed and healing skin, and adjacent normal, intact skin at the wound edges, known as the “periwound.” Maceration of the wound can be as harmful to healing as the lack of sufficient moisture.

Some of the most successful wound care products contain no moisture. Instead they simply reduce the escape of moisture from the wound by being hydrophobic or occlusive. One of the most inexpensive and commonly used occlusive dressings is a fine mesh gauze soaked in petrolatum (i.e. petroleum jelly) infused with an antiseptic chemical (bismuth tribromophenate), commercially available as XEROFORM™ Occlusive Gauze Patch (Covidien, Mansfield, Mass. 02048). XEROFORM™ is used at most burn centers on skin graft donor site wounds.

Skin-grafting is a common and highly effective surgical procedure for reconstructing skin defects caused by traumatic injuries, burns, and surgical excisions where extensive destruction of skin and underlying tissue otherwise could lead to massive scar formation, contractures, disfigurement, and disability. Harvesting of the skin graft also creates a wound, but since this new wound is shallow and superficial it generally heals reliably well, and consequently little attention has been given toward its improved healing.

The clinical consensus among burn surgeons, however, is that donor site wounds are always painful, often more painful than the wounds being treated with the harvested graft. It has been observed that XEROFORM™ treated donor-site wounds are more painful than those that are treated with non-petrolatum-based dressings, but cost, convenience, and tradition militate against using something else.

In the case of severe burns, numerous substances have been shown to benefit the healing process but are expensive to produce and/or inconvenient to handle. These include the use of living cells, either autologous or allogenic.

Advanced wound healing dressings such as amniotic membrane products and human tissue grafts, preferred in treating delayed healing wounds such as diabetic ulcers, are also limited by cost, convenience, and availability.

In recent years, human hair as a biomaterial has become a subject of increasing interest. Various keratin preparations made from hair have shown wound healing efficacy in preclinical studies. Moreover, utilizing abundantly available hair clippings as a starting material solves the logistical problems and shortages related to needing donated human tissue or exotic substances to manufacture advanced wound healing products.

Keratin from feathers and wool also have been considered for this use, and most recently mouse fur was processed into a wound healing preparation. A disadvantage of this preparation is that it is simply a milled powder and not an actual wound dressing.

Most keratin preparations that are proposed for wound healing applications are highly hydrated either by partial oxidation to impart hydrophilicity or by reconstitution from reduced form into a hydrogel. While these hydrogels are useful and efficacious, they lack the feel and “drape” of traditional wound dressings such as gauze and are substantially thicker than membrane dressings derived from tissue such as OASIS® Wound Matrix (Smith & Nephew), made from porcine intestinal submucosa.

Accordingly, a need exists for a wound dressing that delivers moisture to the wound and at the same time protects the periwound from being exposed to excess moisture.

A further need exists for a high-moisture-content dressing that functions such that, as the wound becomes smaller due to healing, the dressing contact area on the skin also becomes smaller along with the size of the wound.

A further need exists for a hydroactive, keratin wound dressing that actively accelerates healing, utilizes a minimal amount of keratin on its wound-contacting surface, has a similar drape and feel of fine mesh gauze, is low cost to produce, and can be packaged in a dry condition and stored at room temperature.

A further need exists for a keratin wound dressing that regulates moisture in the wound, does not contain petrolatum, and contains an antiseptic ingredient.

### SUMMARY

An aspect is a hydrogel film in which evaporation of moisture contained therein causes less shrinkage on the side facing the wound than on the side facing the ambient environment. Thus, a hydrogel film described herein, if placed in open air, will curl up upon drying, but predictably only in one direction. Such a hydrogel film will not curl in contact with an open wound because fluid weeping from the wound will keep the hydrogel fully hydrated and flat. However, due to the dramatically reduced rate of moisture transpiration from intact skin relative to broken skin, as the wound heals, the portion of the hydrogel film now in contact with healed skin will curl upward and lift away from said healed skin, thereby protecting the newly healed skin from exposure to excess moisture and allowing the skin to achieve a healthy status. The wound dressing will thus gradually and spontaneously lift up at its edges and allow healthful circulation of air over the newly-healed skin while continuing to deliver and/or retain moisture where needed on the residual wound. The performance of the described hydrogel film as a wound dressing in response to wound healing is illustrated in FIGS. 1a, 1b, and 1c.

An aspect of this invention comprises a mesh coated with keratin on the wound-contacting side and a hydrogel on the non-wound-contacting side. Said hydrogel may contain therapeutic and/or antiseptic ingredients. Thus, a method is provided to utilize dry keratin, dehydrated keratin hydrogel, or keratin precipitated from solution to create a wound dressing that places keratin in intimate contact with the wound surface while utilizing a minimal amount of said keratin.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1a-c are a series of three illustrations representing in cross-sectional view an aspect.

FIG. 2a is a photograph of the bright, bleeding surface of a superficial wound caused by accidental blunt trauma on the shin of a human subject several days post-injury and immediately upon removal of the resultant scab by gentle debridement.

FIG. 2b is a photograph of the same wound shown in FIG. 2a upon application of a rectangular piece of human keratin matrix, prepared in accordance with Example 1, to a portion of the wound.

FIG. 2c is a photograph showing the appearance of the wound after 8 days; the human keratin matrix having curled up and lifted away from the wound edges, but where not curled remaining firmly attached to the unhealed portion of the wound.

FIG. 2d is a photograph showing the final result of wound healing after 13 days with the human keratin matrix removed.

FIG. 2e is a photograph of the same wound in FIG. 2d upon gentle debridement to remove the scab from the portion of the wound not treated with the human keratin matrix. Note that a small portion of the untreated wound was revealed upon removal of the scab to be unhealed and bleeding.

FIG. 2f is an illustration of the measured changes in wound area upon debridement and reapplication of the human keratin matrix on days 0, 5, and 13 of wound healing.

FIG. 3 is a photograph of a prototype sample of a human keratin matrix in which the word “UP” has been molded into the hydrogel film to instruct the user as to the proper orientation for the applied sample.

FIG. 4a is a photograph of two dressing samples made as described in Example 3 after drying, whereupon the mesh curled toward the gelatin hydrogel-coated side and away from the alginate hydrogel-coated side.

FIG. 4b is a photograph of another sample of the dressing of Example 3 after being placed on a water-soaked piece of paper towel and allowed to dry overnight with the alginate hydrogel-coated side facing down and gelatin hydrogel-coated side facing up. The portion of the dressing covering the wet paper towel remained soft whereas the edges not in contact with moisture dried out and curled up.

FIGS. 5a-c are photographs showing: untreated fine mesh gauze (5a), the same gauze coated with an aqueous emulsion of a flexible binder polymer (5b), and the binder polymer-treated gauze after coating with finely-milled keratin powder (5c).

FIGS. 6a-b are photographs of the sample shown in FIG. 5c after further coating on the non-keratin side with a calcium-crosslinked alginate hydrogel containing 3% bismuth tribromophenate and packaged in a moisture-proof pouch. FIG. 6a shows the hydrogel side of the mesh and FIG. 6b shows the keratin side of the mesh.

FIG. 7a is a photograph of a sheet of silicone rubber having mirror images of the word “UP” etched on its surface.

FIG. 7b is a photograph of the silicone rubber of FIG. 7a in which the etched depressions have been filled with a mixture of calcium stearate and a hydrogel comprising CMC, sodium alginate, and glycerin.

FIG. 8a is a photograph of a substrate comprising a nylon mesh and a crosslinked hydrogel formed by applying the mesh to the silicone rubber of FIG. 7b, coating with a layer of a non-crosslinked hydrogel of CMC, sodium alginate, and glycerin, and curing the sodium alginate component of the hydrogel into calcium alginate by flooding the hydrogel layer with a solution of calcium chloride in aqueous glycerin.

FIG. 8b is a photomicrograph of the nylon mesh component of the substrate of FIG. 8a.

FIG. 9a is a photograph of the substrate of FIG. 8a after drying to convert the hydrogel component into a hydroactive material and placing it “UP” side down on a sheet of PTFE.

FIG. 9b is a photograph of the substrate of FIG. 9a after covering it with an aqueous slurry of purified HPE precipitate.

FIG. 10a is a photograph of the wound-contacting side of the wound dressing obtained upon evaporation of the water component of the keratin slurry-covered substrate of FIG. 9b, which caused the keratin particles to agglutinate and become an adherent film.

FIG. 10b is a photograph of the non-wound contacting side (i.e. the “UP” side) of the wound dressing shown in FIG. 10a.

FIG. 11 is a photograph of a portion of the wound dressing of FIG. 10b that was cut from the sample and placed in a tray of deionized water for one hour, whereupon the colorless, clear, hydroactive layer absorbed water, became swollen, and separated from the non-swollen, translucent, white keratin layer.

FIG. 12 is a photograph of the keratin layer of FIG. 11 that was carefully removed intact, rinsed with deionized water, and allowed to dry into a solid, tan colored ribbon.

FIG. 13 is a photograph of the non-wound-contacting side of a keratin wound dressing made by combining a sheet of the human keratin matrix with a hydrogel layer comprised of calcium alginate, CMC, and glycerin with the “UP”s being raised letters on the hydrogel containing calcium stearate as a pigment.

FIG. 14 is the wound-contacting side (i.e. the human keratin matrix side) of the keratin wound dressing shown in FIG. 13.

### DETAILED DESCRIPTION

### Definition of Terms

The following terms as used herein shall have these defined meanings:

“BME” is 2-mercaptoethanol, also known as beta-mercaptoethanol.

“Clathrate” means a composition comprised of a polymer and a small, non-polymeric molecule in which there exists a strong affinity of the polymer for said smaller, non-polymeric molecule, but in which the two are not covalently bonded to each other.

“CMC” means carboxymethyl cellulose.

“DMSO” is dimethyl sulfoxide.

“Glyceryl polyacrylate” is a clathrate comprised of poly(acrylic acid) as the polymer and glycerin (glycerol) as the associated small molecule.

“HPE” means human hair protein extract.

“HPEC” means human hair protein extract concentrate, which is HPE that has been concentrated by ultrafiltration.

“Hydroactive” material means a substance such as polymer, gel, or mixture of substances having high affinity for water that is either water soluble or water absorptive, but in its hydroactive form is substantially devoid of water.

“Hydrogel” means any non-liquid substance that is uniformly comprised of greater than about 40% water.

“Keratin” means any substance obtained from human hair or animal sources including wool, hair, hooves, fur, and feathers; no matter how crude, refined, processed, fractionated, purified, comminuted, chemically derivatized, blended, copolymerized, or otherwise changed or altered in any way by any means. It also encompasses mixtures of keratin with keratin associated proteins and mixtures of keratin with non-keratin substances that are not readily separable from keratin by practical means.

“Keratin wound dressing” means any product or experimental composition containing keratin that is intended for use in treating a wound to facilitate wound healing.

“Mesh” means any woven, knitted, braided, felted, or non-woven fabric made of any material by any means; any perforated or porous film of any porosity, pore size, composition or thickness; and any backing, substrate, or carrier membrane capable of being coated with the substances described in this specification.

“Periwound” means the margin of skin surrounding a wound that extends approximately one centimeter outward from the wound edge.

“ProgenaMatrix™” is a human keratin matrix manufactured from HPEC.

“PTFE” means polytetrafluoroethylene.

“Shindai” method or process means the conditions and methods utilized to produce HPE.

“Substrate” is the layer or layers of materials upon which a wound-contacting substance is coated or affixed to produce a wound dressing.

“Triacetin” is 1,2,3-triacetoxypropane.

“Wound” means skin that is missing at least its entire epidermal layer and at most is missing all its dermis and no more than about one-centimeter depth of subdermal flesh.

In one aspect, a hydrogel wound dressing having a wound-contact side and a non-wound-contact side may have a rate of moisture evaporation from the non-wound-contact side that is faster than the a rate of moisture evaporation from the wound-contact side. These different rates of evaporation may cause the hydrogel wound dressing to curl toward the non-wound-contact side upon drying.

In the same or a different aspect, the hydrogel wound dressing may include disulfide-crosslinked keratin proteins. In such an aspect, the difference in the rates of moisture evaporation is as a result of a manufacturing process comprising disulfide crosslinkage and gelation of a pre-hydrogel material while the pre-hydrogel material is coated on a surface and exposed to air.

In certain aspects, the hydrogel wound dressing may include an engraving with a mirror image of a message such that if the hydrogel is released from the surface, the engraving becomes a legible feature of the finished hydrogel. For instance, the legible message may be the word “UP”.

The same or a different aspect, the hydrogel wound dressing may include a layer of a non-keratin hydrogel affixed to the non-wound-contact side. For instance, the non-keratin hydrogel may include calcium alginate, CMC, and a plasticizer. The plasticizer may be one or more of glycerin, propylene glycol, glyceryl polyacrylate, and triacetin. Additionally, the non-keratin hydrogel may be cured on a surface comprising an engraving with a mirror image of a message such that if the non-keratin hydrogel is released from the surface, the engraving becomes a legible feature of the released non-keratin hydrogel. For instance, the legible message may be the word “UP.” Further, the non-keratin hydrogel cured on the surface may include a pigment, such as calcium stearate. Further, a therapeutic agent may be included within such a hydrogel layer, where the therapeutic agent may be, for example, one or more of polyhexamethylene biguanide hydrochloride, chlorhexidine gluconate, PVP-iodine, silver, and bismuth tribromophenate.

In the same or a different aspect, the wound-contact side and the non-wound-contact side may include a non-keratin hydrogel and may be bonded together to form a bilayer structure. For instance, the non-keratin hydrogel of the non-wound-contact side may include crosslinked gelatin and the non-keratin hydrogel of the wound-contact side may include calcium alginate. Such a hydrogel wound dressing may include a mesh. Additionally, the hydrogel wound dressing may include a therapeutic agent within a hydrogel layer, where the therapeutic agent may be, for example, one or more of polyhexamethylene biguanide hydrochloride, chlorhexidine gluconate, PVP-iodine, silver, and bismuth tribromophenate.

A process for making a hydrogel wound dressing may include adhering at least one layer of a human keratin matrix (“a first layer”) to at least one layer of either a substrate or a hydrogel (“a second layer”), and compressing the first layer and the second layer until a desired thickness of the first layer and the second layer is obtained.

A process for manufacturing a composite comprising a human keratin matrix includes a) providing a sheet of the human keratin matrix; b) providing a first aqueous solution comprising sodium alginate, CMC, and a plasticizer; c) providing a second aqueous solution comprising a divalent or trivalent salt capable of crosslinking soluble alginate into an insoluble gel; d) providing a surface comprising an engraving with a mirror image of a message that, upon releasing the insoluble gel from the surface, becomes a legible feature of the insoluble gel and filling the engraving with the first solution and a pigment; e) coating the first solution over the surface; f) placing the human keratin matrix over the surface coated with the first solution; g) compressing the surface, the insoluble gel, and the human keratin matrix together until a desired thickness is achieved; h) flooding the human keratin matrix with the second solution and allowing sufficient time for the second solution to permeate through the human keratin matrix and into the first solution coated over the surface to produce the composite; and i) removing the composite from the surface and rinsing the composite with water to remove any excess second solution on the composite. Additionally, a mesh may be placed over the surface after coating the first solution over the surface and additional first solution may be coated over the mesh.

Prior to the demonstration of wound healing efficacy of the powdered material noted above, it was believed that the wound healing efficacy of keratin is enabled only upon conversion from its natural state (i.e. hair, wool, fur, and feathers) into a more highly hydrated form. Enzyme-catalyzed reactions that take place in the human body require the presence of water. Yet, while not wishing to be bound by any theory, it is now believed that keratin in a powdered form, no matter how dry, is likely susceptible to a finite degree of hydration such that enzyme-mediated degradation in a moist wound environment is sufficient to release wound-healing factors from the keratin. Thus, wound dressings including keratin may be beneficial for encouraging wound healing.

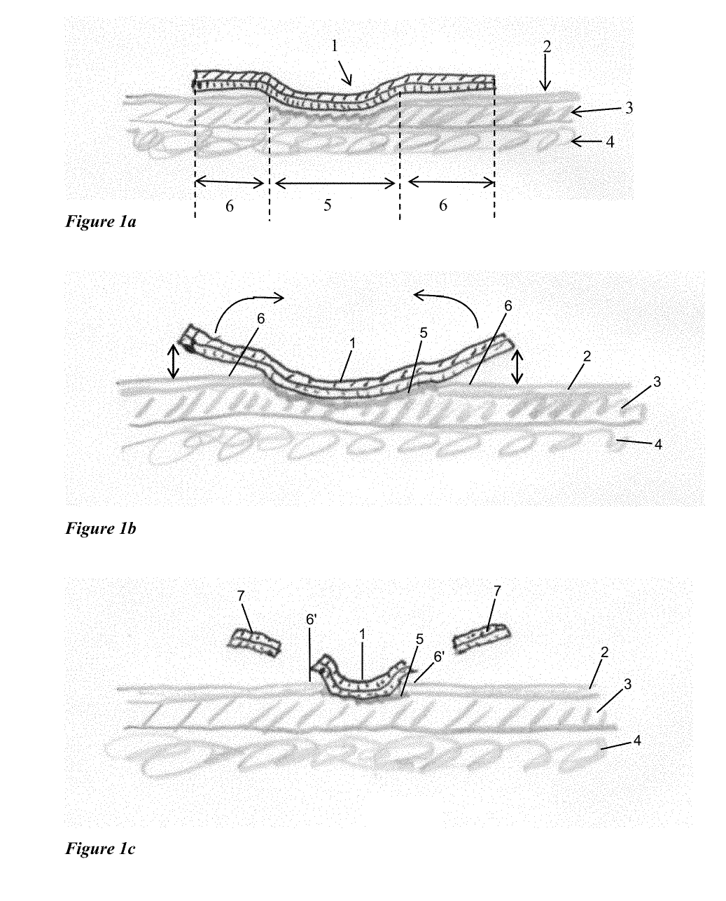

FIGS. 1a-c provide three illustrations of a cross-sectional view of an aspect of the skin-protective hydrogel wound dressing 1 applied to a wound 5 that has eroded through the epidermis 2 and into the dermis 3, but not into the underlying fat 4. The wound 5 and periwound 6 areas are both initially covered by the skin-protective hydrogel wound dressing 1. The skin-protective hydrogel wound dressing 1 soon curls upward and lifts off the periwound 6 as illustrated in FIG. 1b, the moisture transpiration from the periwound 6 being lower in comparison to the wound 5, which may be weeping fluid. As illustrated in FIG. 1c, healing and gradual closure of the wound 5 forms new periwound 6 that, in turn, becomes uncovered as the skin-protective hydrogel wound dressing 1 continues to lift off the epidermis 2. Excess dry skin-protective hydrogel wound dressing 1 is brittle and breaks off or is intentionally trimmed as indicated, to give dressing fractions 7.

Described are wound dressings that are useful in maintaining a moist wound environment while simultaneously protecting newly healed skin and periwound from harmful maceration.

One aspect of a method of making the human keratin matrix that imparts the desired unidirectional curling attribute is accomplished by coating HPEC onto a sheet of medical grade silicone rubber and allowing evaporation and air oxidation to proceed in a controlled manner. Once the HPEC is converted from a liquid into a gel, the coated silicone rubber is immersed in denatured ethyl alcohol. The hydrogel film releases then from the silicone rubber. Next. the hydrogel film is sequentially soaked in 50% alcohol followed by copious deionized water to remove processing chemicals formerly present in the HPEC.

The resultant human keratin matrix can be cut into desired sizes, sealed in moisture proof pouches, and sterilized by radiation. Upon removal from the pouch, the hydrogel film begins to dry and curl within about 5 minutes. At first it was assumed that the curling was occurring toward the side of the film that was in contact with air during curing and not the side that was in contact with silicone rubber during curing. Logically the air side would be more exposed to oxygen, which accelerates reformation of disulfide linkages formerly present in the keratin proteins prior to reductive cleavage during the production of HPEC. Surprisingly, the curl in fact was observed to occur toward the opposite side. By way of example, the “curl” can be defined as the amount of hydrogel film not in direct contact with the patient (i.e., the skin, or wound, of the patient). It is noted that this curling effect happens over time, with 90-100% of the hydrogel film in contact with the patient initially, with the “curl” happening over time. The “curl” can therefore be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 80, 82, 83, 84, 85, 86, 87, 88, 89, 90, 91, 92, 93, 94, 95, 96, 97, 98, or 99%, or any fractional part thereof, of the hydrogel which is not in direct contact with the patient because it is curling upward, away from the body of the patient. The curling effect can take place in 8-120 hours (i.e., in 8, 16, 24, 32, 48, 60, 72, 84, 96, 908, 120, or more hours, or any fractional part thereof, or in 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more days, or any fractional part thereof).

That the direction of the curl is toward the silicone side rather than the air side affords a practical advantage in manufacturing the human keratin matrix since it facilitates the creation of markings that prevent confusion regarding the side that is to be applied on the wound. If the wrong side is placed on the wound, the film will curl downward instead of upward and dig into the skin that it was designed to protect. Thus, a mirror image of the word “UP” can be engraved into the silicone rubber such that liquid HPEC flows into the engraved recessions and the resultant product separated therefrom has the word “UP” clearly visible thereon, as shown in FIG. 3.

The rapid drying and curling of the human keratin matrix upon removal from its moisture-proof packaging necessitates immediate application on the wound. However, in some instances such rapid use is not convenient, for example in the case where the product must be carefully cut to size or an intervening, distracting task necessitates delayed application. To extend the time between removal from package and the onset of drying and curling, a suitable humectant can be added to the water contained in the package with the human keratin matrix. Suitable humectants useful in delaying the onset of drying include glycerol, propylene glycol, triacetin, and glyceryl polyacrylate.

Another method of moderating the rate of drying and curing of the human keratin matrix is to coat the non-wound-contact side with a second hydrogel possessing a slower rate of drying. A preferred hydrogel for this purpose includes calcium alginate and CMC, plasticized with either glycerol or propylene glycol. In this case, the “UP” lettering is preferably created in the alginate gel rather than in the human keratin matrix. One means of bonding alginate-CMC gel to the human keratin matrix is to coat a sodium alginate-CMC formulation onto the silicone, cover the layer of sodium alginate-CMC with a sheet of the human keratin matrix, compress the layers, and then provide a divalent or trivalent cation-containing solution (e.g., a calcium chloride solution) in contact with the human keratin matrix surface until the calcium ions permeate through the human keratin matrix layer into the sodium alginate layer and convert the sodium alginate (non-crosslinked, water soluble) into insoluble, crosslinked calcium alginate.

Although this aspect uses a human keratin matrix with the word “UP” molded into the secondary hydrogel film, non-keratin materials may also be used, as described next.

One such aspect is a hydrogel film wound dressing containing two layers of hydrogel that are bonded together, the top layer being faster drying than the bottom (wound contacting) layer. Neither of these layers need to be formed from a keratin-containing material. By “faster drying” is meant that the top layer is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 80, 82, 83, 84, 85, 86, 87, 88, 89, 90, 91, 92, 93, 94, 95, 96, 97, 98, or 99%, or any fractional part thereof, faster drying compared with the bottom layer. A wide variety of hydrogel polymers are available with which to create such a bilayered structure, many of which are described by Dutta, et al. in PCT Patent Publication WO/2010/067378 entitled, “Hydrogel Composition,” the teachings of which are incorporated herein. In addition, numerous examples of wound dressings comprising multiple layers of varied materials have been previously described

In general, any film-forming hydrogel composition in which the crosslink density can be modified will be suitable. For example, sodium alginate can be crosslinked with calcium ions, as described by Patel in U.S. Pat. No. 5,470,576 entitled, “Process for preparing the alginate-containing wound dressing,” the teachings of which are incorporated herein. Another example of a method of creating a calcium crosslinked alginate dressing is described by Barrows in U.S. Patent Publication No. 2014/0200196, the teachings of which are incorporated herein.

Calcium alginate hydrogels, being ionically, rather than covalently, crosslinked, have an advantage of being able to absorb large amounts of wound exudate. As the crosslink density is reduced, the capacity of the gel to hold water increases. On the other hand, as exudate production diminishes alginate dressings shrink as they dry, but generally remain adherent to the wound and need to be removed by physically cleansing the wound.

Another type of non-covalently crosslinked hydrogel wound dressing includes glyceryl polyacrylate. As mentioned above, glyceryl polyacrylate is a clathrate hydrogel that has a tenacious affinity for retained water and is very slow to dry out. Thus, a bilayer hydrogel of calcium alginate and glyceryl polyacrylate represents a non-keratin aspect.

Further aspects include addition of a layer of fabric or gauze to impart strength to the dressing and the incorporation of therapeutic substances such growth factors, analgesics, anti-inflammatory agents, antibiotics, and antimicrobials.

Related aspects feature a mesh upon which keratin is thinly coated. In this way, the physical properties of the mesh dominate the outward appearance, feel, and handling of the wound dressing while its healing efficacy is enhanced by an interface between keratin and the open wound. In this configuration the keratin wound dressing may also feature a non-keratin hydrogel or hydroactive layer on the opposite side of the keratin-coated mesh.

In one aspect, particles of keratin are attached to a mesh by first priming the mesh with an adhesive substance. In the art of non-woven technology, binder polymer formulations having a wide variety of performance characteristics are well known and utilized in the manufacture of numerous consumer products. For example, one binder is an emulsion polymer formulated to yield a flexible bond between the components needing to be bonded such that minimal stiffness is added to the resultant article. An exemplary emulsion polymer is DUR-O-SET™ (Celanese Corp.).

An example of a pure keratin hydrogel wound dressing is a human keratin matrix, as discussed previously, which is about 300 μm thick.

The keratin layer of the wound dressing may be about 5 μm to about 300 μm or more, or about 5 μm, 10 μm, 20 μm, 50 μm, 100 μm, 150 μm, 200 μm, 250 μm, 300 μm, or 500 μm thick (or more), or any fractional part thereof. The total dressing can be from 0.1 mm to 5.0 mm in thickness. In accordance with the principles disclosed herein, most of the thickness of the human keratin matrix need not be keratin. It could instead be formed mostly of a lower cost hydrogel; one that would accomplish the generic benefits of maintaining an optimally moist wound healing environment without otherwise having any biological activity.

An important advantage of the human keratin matrix having an added hydrogel layer is that the rate and extent of curling (lifting of the edges of the dressing as the wound heals, as discussed previously) can be controlled and adjusted. Thus, a thick coating of hydrogel may eliminate the curl-up feature of the human keratin matrix while progressively thinner coatings of hydrogel will restore that feature in gradations, allowing the magnitude of this attribute to be adjusted to suit specific wound treatment situations.

In one aspect, it is envisioned that a mesh, e.g., a fine mesh gauze such as the one comprising XEROFORM™, can serve both as a mechanical reinforcement support and as a partition between keratin (in any form) on the wound-contacting side and a low-cost hydrogel on the opposite side. In certain aspects, the mesh is woven nylon fabric having about 70 μm pore openings and a weight of about 3 mg/cm², commercially available as 3M TEGADERM™ Non-Adherent Contact Layer (3M Company, 3M Center, St. Paul, Minn. 55144).

Exemplary mesh openings can be from 5 μm to 2 mm or larger. In certain aspects, types of mesh that can be used include, but are not limited to, ADAPTIC TOUCH™ Non-Adhering Silicone Dressing; N-TERFACE® Wound Contact Material; CARDINAL HEALTH™ Petrolatum Emulsion Contact Layer; CARDINAL HEALTH™ Silicone Contact Layers; COLACTIVE® Transfer (Wound Contact Layer); COMFITEL™ Silicone Contact Layer Dressing; CONFORMANT®2 Non-Adherent Contact Layer; COVRSITE® Wound Cover; CUTICELL® Contact; DERMANET®; DRYNET* Wound Veil; MEPITEL® One; MEPITEL® Soft Silicone Wound Contact; PHYSIOTULLE® Wound Contact Layer; PROFORE® Wound Contact Layer Non-Adherent Dressing; RESTORE® Contact Layer FLEX; SILFLEX® Soft Silicone Wound Contact Layer; SILON-TSR®; TELFA™ Clear; and TRITEC™.

This mesh-plus-hydrogel configuration may allow a therapeutic agent or antiseptic ingredient to be blended into the hydrogel component. Thus, a relatively thick layer of hydrogel on the non-keratin side of the mesh facilitates “loading” a large amount of said agent or therapeutic into the “delivery vehicle” (i.e. hydrogel). By “relatively thick” is meant up to about 5 mm thick. By “fine mesh” is meant having mesh openings that are from about 50 μm to about 2 mm in size. Exemplary antiseptics include polyhexamethylene biguanide hydrochloride, chlorhexidine gluconate, PVP-iodine, and silver sulfadiazine. A preferred antiseptic is bismuth tribromophenate.

As illustrated in Example 5 below, a calcium alginate hydrogel can be used to create a product that resembles XEROFORM™, but without the bismuth compound needing to be infused into petrolatum, an objectionable substance, as described above. The substitution of petrolatum with hydrogel as a vehicle for bismuth tribromophenate on a mesh such as fine mesh cotton gauze is anticipated to provide wound dressing products, with or without keratin, that provide improved patient comfort during the healing of skin graft donor site wounds.

A wide variety of polymers are available with which to create the non-keratin hydrogel component, many of which are described by Dutta, et al. in PCT Patent Publication WO/2010/067378 entitled, “Hydrogel Composition”, the teachings of which are incorporated herein.

An example of a crosslinked alginate dressing is described by Patel in U.S. Pat. No. 5,470,576 entitled, “Process for preparing the alginate-containing wound dressing”, the teachings of which are incorporated herein. Another example of a method of creating a calcium crosslinked alginate dressing is described by Barrows in U.S. Patent Publication No. 2014/0200196, the teachings of which are incorporated herein.

In the same or a different aspect, a wound dressing may have a wound-contact side and a non-wound-contact side. The wound-contact side may include keratin in a non-hydrogel state and the non-wound-contact side may include a non-keratin hydrogel. In the same or a different aspect, the keratin may include at least one layer of agglutinated particles. The non-keratin hydrogel may include a mesh. Additionally, a therapeutic agent may be included within the hydrogel layer, where the therapeutic agent may be one or more of polyhexamethylene biguanide hydrochloride, chlorhexidine gluconate, PVP-iodine, silver, and bismuth tribromophenate.

A process of forming a human keratin matrix includes: mixing an aqueous solution of keratin with a mater-miscible organic solvent to obtain keratin particles; collecting the keratin particles; re-suspending the keratin particles in water to produce a keratin slurry; applying the keratin slurry to a substrate; and drying the substrate with the keratin slurry so as to bond the keratin particles of the keratin slurry to the substrate.

In the same or a different aspect, a process of forming a wound dressing includes: a) mixing an aqueous solution of keratin with a water-miscible organic solvent to obtain keratin particles; b) collecting the keratin particles and re-suspending the keratin particles in water to produce a slurry of keratin particles; c) providing a substrate; d) spreading the slurry of keratin particles on the substrate; and e) drying the substrate to agglutinate the keratin particles and to bond the agglutinated keratin particles to the substrate.

The invention is further described by the following examples that are provided for illustration, not limitation.

### Example 1. Human Keratin Matrix

Approximately 4 milliliters of dimethyl sulfoxide were combined with 6 milliliters of HPEC as prepared according to U.S. Pat. No. 9,072,818 (Example 1), incorporated herein by reference, and mixed for about 10 minutes at which time an increase in the viscosity of the solution was observed. The resultant viscous solution of DMSO/HPEC was then poured onto a sheet of medical grade NS-51 translucent silicone rubber within an 8-cm inside diameter ring of the same silicone rubber to confine the pool of liquid within an 8-cm diameter circle. The surface of the silicone rubber under the pool of DMSO/HPEC was previously engraved with the mirror-image of the word “UP” by laser ablation to a depth of approximately 150 μm. The DMSO/HPEC pool was left undisturbed in a well-ventilated chemical fume hood for approximately 3 hours, whereupon it gradually became more viscous, tacky, and then a solid gel. Watchful monitoring prevented excessive drying, which otherwise would cause crystallization of the highly concentrated urea present in the HPEC. The addition of DMSO to HPEC, in addition to causing a viscosity increase, was discovered to suppress the crystallization of urea.

The silicone rubber sheet with its coating of HPEC gel was then immersed in a beaker of denatured ethyl alcohol and allowed to soak for about one hour, at which time the cured HPEC gel disc was easily removable from the silicon rubber, revealing the molded word “UP” as shown in FIG. 3. The disc of HPEC gel was then placed in a beaker of a 1:1 mixture by volume of denatured alcohol/water and allowed to soak overnight. The disc was then transferred to a beaker containing one liter of deionized water, which was replaced with fresh deionized water 6 times over the course of 3 days. These soaking steps removed virtually all undesired chemical species originally present in the HPEC to yield a pure keratin protein hydrogel.

With care not to allow any drying of the hydrogel to take place, the gel film was trimmed to a 6-cm diameter disc to remove edge imperfections from the original 8-cm disc and placed in a heat-sealable, moisture-proof pouch containing about 5 mL of a 20% (v/v) solution of propylene glycol, USP, in water to ensure maximum hydration of the human keratin matrix during storage. The propylene glycol serves as a humectant. (Note that aqueous glycerol, triacetin, and/or glyceryl polyacrylate also could be used in substitution for propylene glycol as humectants). The pouch was then heat-sealed and exposed to 25 kGy of electron beam radiation to sterilize the contents.

### Example 2. Use of Human Keratin Matrix in Healing a Wound

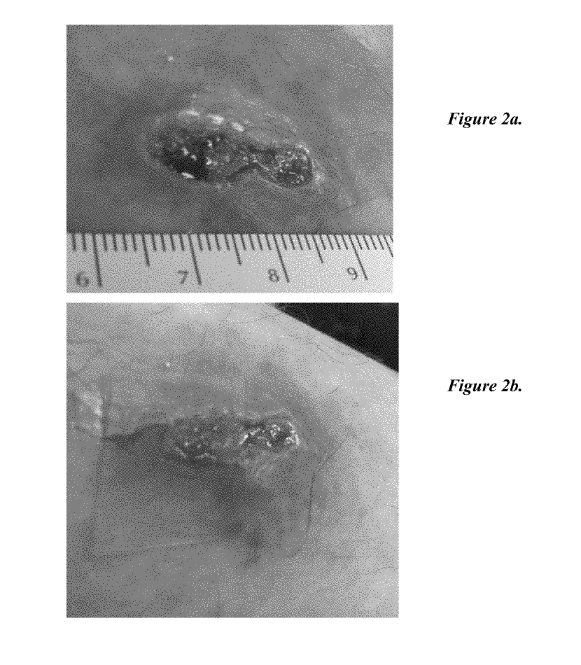

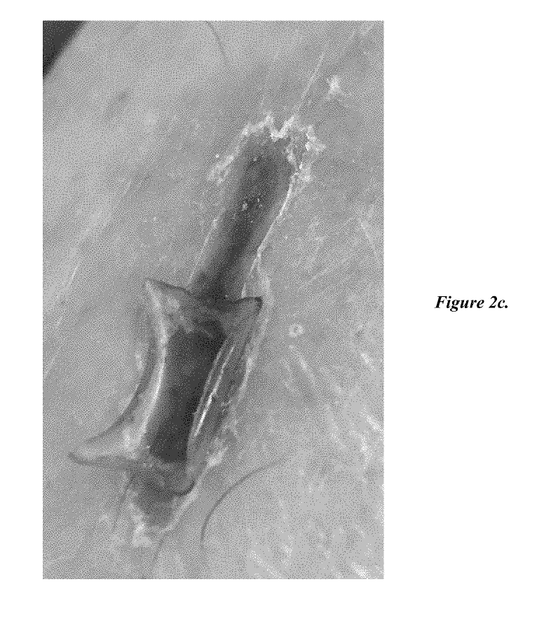

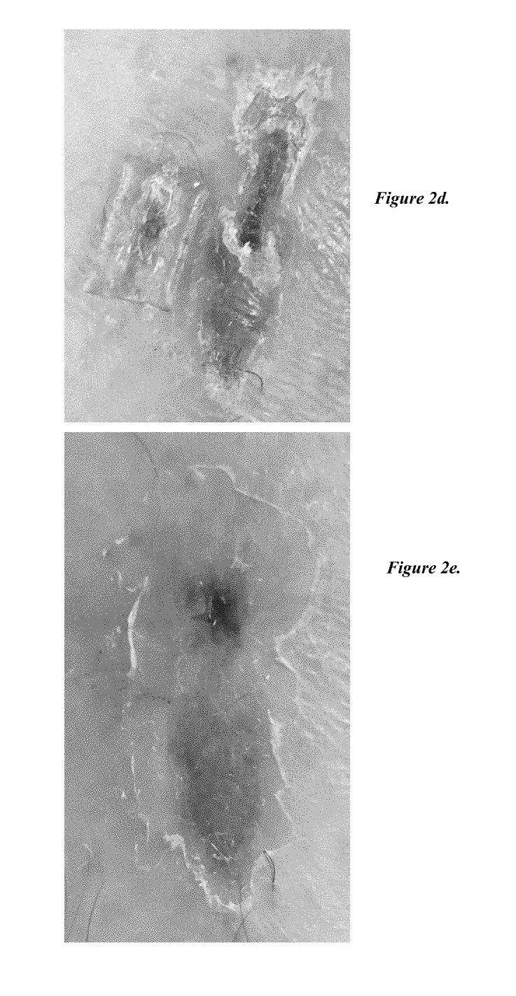

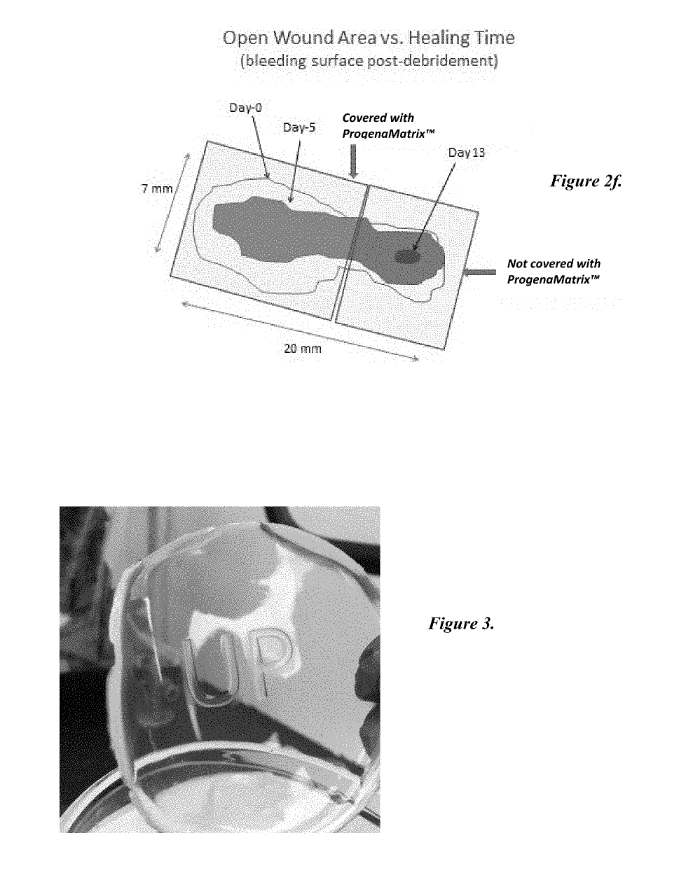

A 69 year old male volunteer presented with a 3-day old, fully scabbed wound resulting from accidental blunt trauma sustained on the shin. The scab was removed by gentle debridement with sterile saline-soaked gauze to reveal a bright, bleeding wound with surrounding inflamed skin as shown in FIG. 2a. A small rectangular piece of sterile human keratin matrix as described in Example 1 was applied directly to a portion of the wound taking care to ensure that the “UP” side was facing up, then covered with a BAND-AID® brand adhesive bandage. The portion of the wound not covered with the human keratin matrix was also covered with a BAND-AID®. On day 5 post-treatment the dressings were removed and the wound again debrided to allow measurement of the healed and non-healed areas, followed by re-application of fresh dressings exactly as on day 0. On day 13 the dressings were again removed and the wound debrided to measure healing. At this time only a small spot of open wound was observed in the non-human-keratin-matrix-treated area.

The curl-up feature of the human keratin matrix is clearly shown in FIG. 2c, which was taken on day-8. Note that all four edges of the original rectangular piece of the human keratin matrix have curled upward and lifted away from the periwound, whereas the central portion, continuing to be in intimate contact with the residual wound, remained flat. On day-13, sufficient healing under the human keratin matrix had taken place such that it essentially fell off. As shown in FIG. 2d, the portion of the wound under the human keratin matrix did not have a scab and thus healed by the mechanism attributed to optimal moist wound healing, whereas the portion treated with BAND-AID® only remained covered with a scab. Subsequent debridement of the wound (FIG. 2d) revealed the final result shown in FIG. 2e. Healing under both the human keratin matrix and the scab was almost equally complete except for a small spot of residual bleeding seen under the scab.

A collage of unhealed wound area measurements at each post-debridement timepoint is shown in FIG. 2f. Note that the human keratin matrix treated portion of the wound underwent a noticeably greater reduction in wound area (i.e. faster healing) than the BAND-AID® only treated portion of the wound.

### Example 3. Hydrogel Bilayer Wound Dressing with Curl-Up Periwound Protection Feature

A solution of gelatin was prepared by mixing an envelope (7.2 g) of KNOX® gelatin (Kraft Foods North America Division of Kraft Foods Global, Inc, Tarrytown, N.Y. 10591) with 250 mL of cold tap water in a beaker and heating in a microwave oven until hot. The mixture was stirred for a few minutes to dissolve and then allowed to cool to room temperature.

A single ply of medical grade gauze fabric (Johnson & Johnson QUILTVENT® BAND-AID® 4×4″ sterile pad, J&J Consumer Companies, Inc., Skillman, N.J. 08558) was placed in a 7×7″ PYREX® glass baking dish and the gelatin solution was poured onto the gauze until completely soaked. Excess gelatin solution was decanted from the dish, which was then placed in a refrigerator to gel overnight.

A coating of alginate-based hydrogel liquid (SKINLOCK™ transparent tattoo sealant, Tattoo Innovations, Inc., Oakville, ON Canada L6J 3Z7) was manually applied to the surface of the gelatin/gauze composite and then misted with the calcium chloride spray supplied with the SKINLOCK™. This caused the alginate to cure into a crosslinked hydrogel.

The gel-impregnated gauze was removed from the PYREX® dish and replaced with a solution of 2.5% (v/v) glutaraldehyde prepared by mixing 45 mL of deionized water with 5 mL of a 25% solution of glutaraldehyde (Sigma-Aldrich product no. G6257, batch no. MKBD547). The gel/gauze was then carefully laid back down into the dish of glutaraldehyde and allowed to soak for one hour, after which time it was removed, rinsed with water and the allowed to soak in fresh water for several hours. Samples for further use were cut from this piece with scissors, sealed in moisture-proof pouches, and placed in the refrigerator for storage.

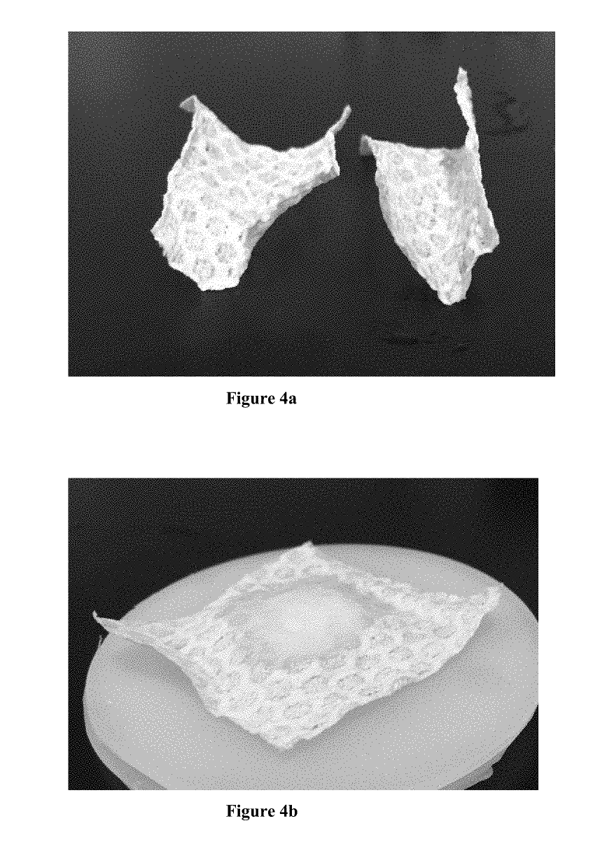

Samples of this material were allowed to dry overnight, whereupon they curled toward the gelatin side and away from the alginate side. This observation, shown in FIG. 4a, was attributed to differences between the two sides with respect to moisture retention and crosslink density, glutaraldehyde crosslinked gelatin presumably having less of the former and more of the latter. FIG. 4b illustrates the curling that occurred upon overnight drying with a water-saturated piece of paper towel under the sample, which kept that region of the gel-impregnated gauze soft and non-curled.

### Example 4. Fine Mesh Cotton Gauze Coated with a Finely-Milled Powder of Human Hair Keratin

The human keratin matrix made in Example 1 was allowed to dry completely in open air. Approximately 50 grams were crushed and coarsely ground in an electric coffee bean grinder and then shipped to a contract manufacturing facility for micronization via jet-milling. The resultant powder had a soft, smooth feel with a reported average particle size of about 15 μm.

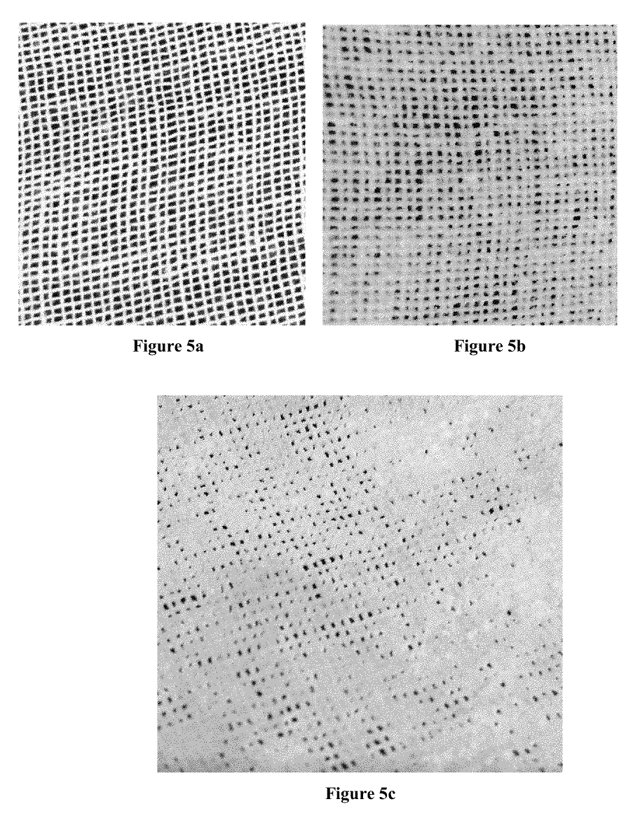

A sample of fine mesh cotton gauze (DeRoyal Industries, Inc., Powell, Tenn. 37849, Product No. 10-1818) was soaked with an emulsion of a flexible polymer typically used in the manufacture of non-woven products (DUR-O-SET® Elite Ultra, Product No. 25-135A, Celanese Emulsion Polymers, Celanese Corp.) and blotted to remove excess liquid. The keratin powder was then applied to the tackified mesh, excess powder dusted off, and the resultant powder-coated mesh allowed to dry. The photographs of FIG. 5. show the original untreated mesh (FIG. 5a), the mesh after coating with binder polymer (FIG. 5b), and the keratin powder-coated mesh after removal of excess powder and drying (FIG. 5c).

### Example 5. Keratin-Coated Mesh with Antiseptic-Infused Hydrogel Backing

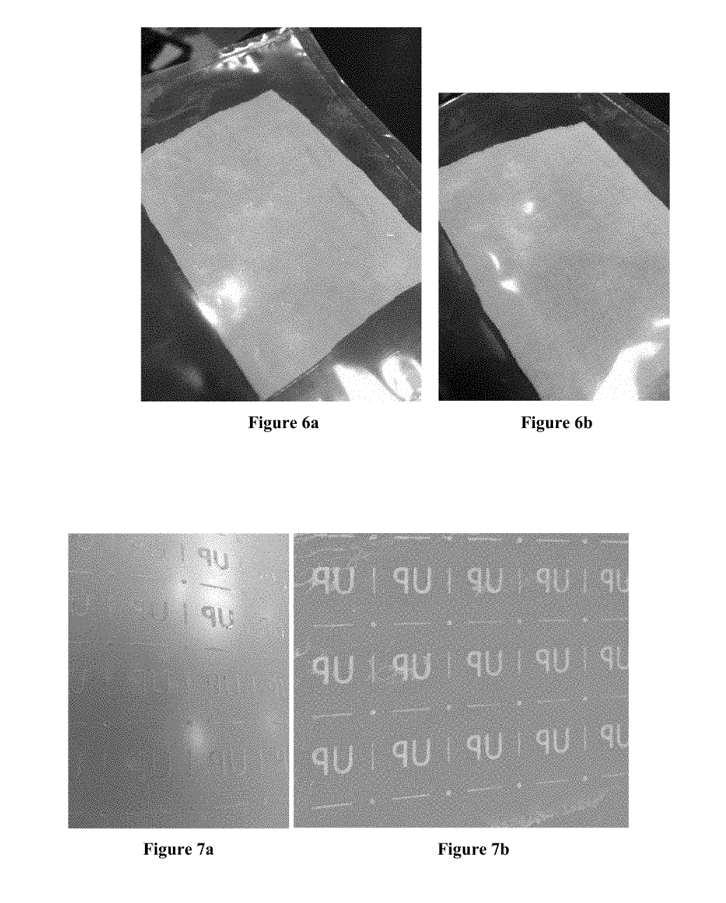

A commercially available alginate polymer-based kit for in-situ formation of calcium-crosslinked hydrogel film was utilized in this Example (SKINLOCK™, Tattoo Innovations Inc., Oakville, ON, Canada, L6J 3Z7), comprising a spreadable gel (aqueous sodium alginate and other polymers) and a spray bottle of calcium chloride solution. A sample of the viscous alginate gel component (9.70 grams) was placed in a 15-mL conical tube and mixed with 0.30 grams of bismuth tribromophenate (Spectrum Chemical Co., Product No. B1121, lot no. 2CE0057) to obtain a 3% (w/w) mixture, which is the same percent of this antiseptic contained in XEROFORM™. The mixture was then thoroughly blended with the use of a rotor-stator electric homogenizer (OMNI-TH Homogenizer, Omni International Inc., Kennesaw, Ga. 30144) to yield a blend having uniform yellow color and a creamy consistency. The gel was spread out on a sheet of heavy, food-grade silicon rubber to provide a thin layer (approximately 3 mm thick), upon which was placed the keratin-coated mesh of Example 4, keratin-coated side up. It was then sprayed with calcium chloride solution using the spray product supplied with the SKINLOCK™ kit. This caused instant curing of the alginate gel into a crosslinked film, allowing the mesh and hydrogel film to be easily peeled off the silicone rubber. It was then soaked in water to remove excess calcium solution and sealed in a moisture-proof pouch, as shown in FIGS. 6a (alginate gel side) and 6b (keratin powder-coated side).

### Example 6. Fabrication of a Wound Care Dressing Substrate

A 25% (v/v) solution of glycerin was prepared by combining 250 mL of glycerin (Spectrum Chemical Co. product no. G1016) and 750 mL of deionized water in a beaker with magnetic mixing until clear. 5.00 grams of sodium alginate (Spectrum Chemical Co. No. S1118) were placed in an 8-oz. screw cap jar and the jar filled with the above glycerin solution, mixed, capped, and the alginate allowed to hydrate overnight during storage at 4° C. in a refrigerator. Likewise, 15.00 grams of CMC (Spectrum Chemical Co. product no. CA194) were placed in a separate 8-oz. screw cap jar and the jar filled with glycerin solution, mixed, capped, and the CMC allowed to hydrate overnight. The contents of the jars were then transferred into a 4.5-quart stainless-steel bowl, combined with the remaining prepared 25% glycerin solution and stirred with a wire whisk agitator using a mixer. Stirring was continued at slow speed until all lumps dissolved to produce a thick, smooth, clear gel.

About 30 grams of the above gel were placed in a 4-oz. glass jar and combined with about 3 grams of calcium stearate powder (Spectrum Chemical Co. product no. CA169) and mixed by hand with a spatula until well blended to give an opaque white gel. A ¼″ thick slab (17 cm×31 cm) of medical-grade silicone rubber having laser-etched mirror-image depressions of the word “UP” was used as a surface upon which to mold the gel, shown in FIG. 7a. A small amount of the white calcium stearate gel was manually rubbed into the “UP” depressions on the mold and excess gel carefully wiped off, leaving the depressing filled with white gel, as shown in FIG. 7b.

A 7.5×20.1 cm sheet of 3M TEGADERM™ Contact Non-Adherent Contact Layer nylon mesh (3M Health Care, St. Paul, Minn. 55144) was placed on a paper towel and coated with the clear alginate/CMC gel by manually spreading and smoothing. This was then placed with the gel side down on top of the silicone mold having white gel in the lettering depressions. The surface of the mesh was gently smoothed to produce uniformly thick layer of gel between the mesh and the silicone rubber.

A solution of 10% w/v calcium chloride dihydrate in 25% v/v glycerin was made by adding 10.0 grams of calcium chloride dihydrate (Sigma-Aldrich product no. 223506) to a 100 mL graduated cylinder containing about 60 mL of deionized water while stirring with a spin bar on a magnetic mixer. Upon dissolution of the calcium chloride the spin bar was retrieved, and additional deionized water added to the 75 mL mark. Glycerin (Spectrum Chemical Co. product no. G1016) was added to bring the liquid level up to the 100 mL mark. The liquid was then poured into a beaker and mixing continued for a few minutes, and then poured onto the surface of the mesh such that the entire mesh was flooded with liquid. This was left undisturbed for about one hour to allow the calcium to permeate the alginate/CMC gel and convert the sodium alginate into calcium alginate.

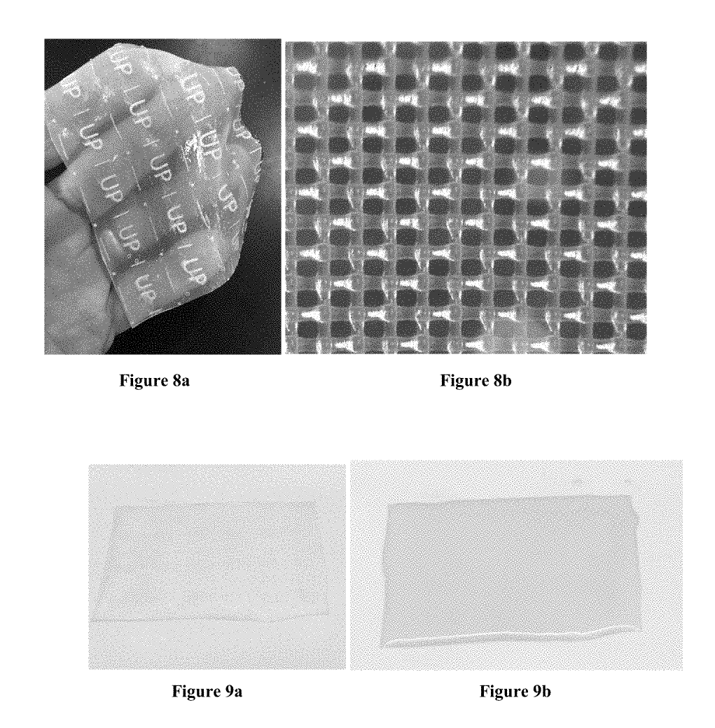

The mesh was removed from the silicone, taking care not to disrupt the white gel contained within the depressions and incorporated into the calcium alginate gel. The appearance of the gel-impregnated mesh upon removal from the mold is shown in FIG. 8a and a magnified view of the mesh alone is shown in FIG. 8b. Note that the mesh, being a colorless nylon monofilament fabric, virtually disappeared within the gel to yield a transparent substrate, except for the white lettering produced by the calcium stearate content of the gel within the mold cavities. The gel-impregnated mesh was then rinsed with deionized water and allow to dry in air for several hours. It was then further dried to constant weight in a food dehydrator at 95° F. The drying process removed water but retained glycerin, which acted as a plasticizer to create a hydroactive composition that was soft and flexible. Based on before-and-after measurements of weights and dimensions, the resultant substrate composition was determined to be 3.3 mg/cm² nylon mesh and 30.9 mg/cm² hydroactive material.

Although glycerin was used as the plasticizer and humectant in this Example to obtain a hydroactive substrate, other such plasticizers could serve this purpose equally well included, for example, propylene glycol, glyceryl polyacrylate, and triacetin.

### Example 7. Fabrication of a Keratin Wound Dressing from HPE Precipitate

A kitchen blender was filled with about 1.4 liters of denatured ethyl alcohol and the motor started on the “blend” setting. HPE (100 mL) was slowly poured into the rapidly mixing alcohol to precipitate the dissolved keratin. After about one minute the mixing was stopped, and the precipitate allowed to coagulate and settle. About ⅔ of the liquid was decanted and the precipitate collected in a funnel lined with filter paper. The solids were then resuspended in a mixture of 500 mL denatured alcohol and 500 mL of deionized water and again subjected to blender mixing for about one minute. The decanting and filtering steps were repeated, and the collected precipitate placed in ajar, which was then tightly sealed and stored in the refrigerator overnight.

Some water that separated from the solids was poured out and about 15 mL of the purified precipitate was placed in a graduated 50 mL beaker and deionized water added to the 30 mL mark. A spin bar was added, and the mixture stirred rapidly for about one hour, which converted the precipitate into a creamy suspension.

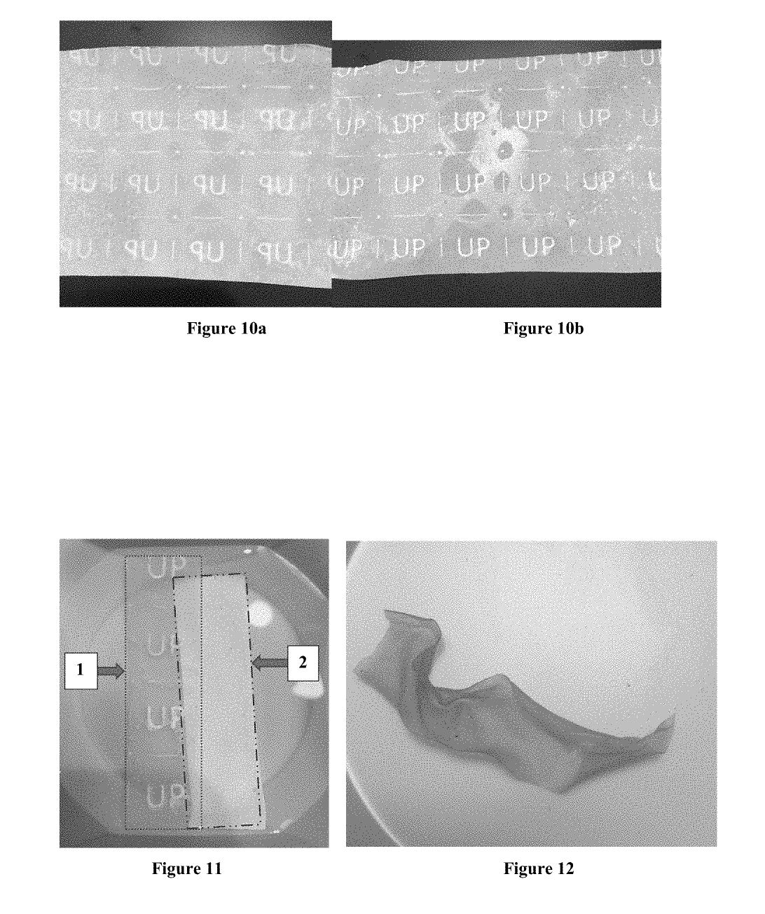

The substrate of Example 6 was placed up-side-down on a sheet of PTFE (FIG. 9a) and the above suspension poured over the surface, as shown in FIG. 9b, and allowed to dry for several hours. It was then further dried to constant weight in a food dehydrator at 95° F. The resultant dry membrane, as shown in FIG. 10a (wound contact side up) and FIG. 10b (wound contact side down) is a soft, flexible, translucent membrane with excellent drape and feel.

### Example 8. Determination of the Keratin Content of the Wound Dressing of Example 7

A portion of the wound dressing of Example 7 was cut from the sample, measured, and weighed. It was then submerged in a tray of deionized water and allowed to soak for one hour, during which time the hydroactive material became hydrated and swollen, causing the substantially less hydrated keratin layer to separate and slide off, as shown in FIG. 11. The keratin layer was carefully removed intact, rinsed with deionized water, and allowed to dry in open air for several hours, then further dried in a dehydrator at 95° F. to a constant weight. The separated keratin layer upon drying was a tan colored and brittle material (FIG. 12) suggesting that being laminated to the hydroactive material imparted softness and some degree of retained hydration and/or possible plasticization. The determined weights of the component layers are listed below:

| Layer | Weight (mg/cm²) |
|---|---|
| Mesh | 3.3 |
| Keratin | 5.4 |
| Hydroactive | 30.9 |

### Example 9. Human Keratin Matrix with Hydrogel Layer

A 9.5×9.5 cm piece of human keratin matrix was removed from the package, rinsed with tap water, blotted dry and weighed to reveal a weight of 52 mg/cm². The silicone mold of Example 6 was again coated with the described alginate gel-calcium stearate mixture to fill the depressions as shown in FIG. 7b. About 30 mL of the alginate-CMC gel of Example 6 was heated to 70° C., placed in the center of the mold, and covered with the human keratin matrix with its “UP” side down. Wooden tongue depressor blades were placed on all four sides to serve as spacers to control thickness of the gel layer. This assembly was then covered completely with a sheet of polyethylene film (GLAD® Cling Wrap, Glad Products Co., Oakland, Calif. 94612). A heavy slab of PTFE was placed on top and pressed until flush with the spacers. The PTFE and polyethylene film were removed and a solution of calcium chloride in 25% glycerin was poured over the top of the human keratin matrix and surrounding excess gel and allowed to soak for about 30 minutes.

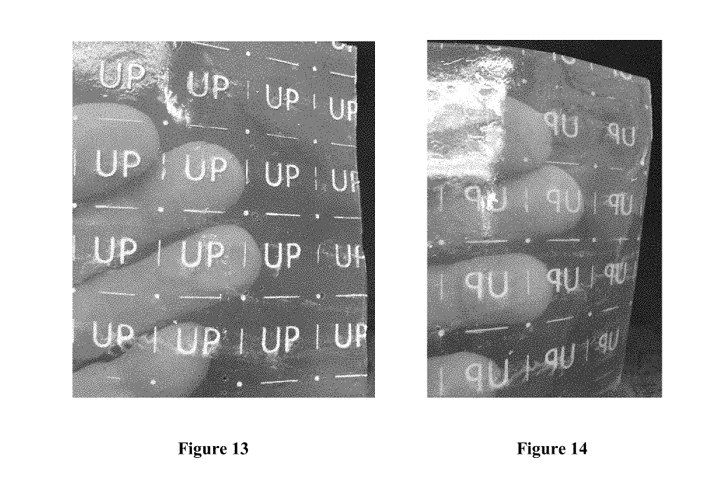

Upon removal from the mold, the resultant bilayer of human keratin matrix was rinsed with tap water, excess gel trimmed, and weighed. By subtraction of the original weight of human keratin matrix it was determined that the gel coating weight was 58 mg/cm². As shown in FIG. 13 (non-wound-contact side) and FIG. 14 (wound-contact side), this dressing is clear and flexible with highly visible markings to indicate the correct side that should be applied to the wound.

While the invention has been illustrated by a description of various aspects and while these aspects have been described in considerable detail, it is not the intention of the applicant to restrict or in any way limit the scope of the appended claims to such detail. Additional advantages and modifications will readily appear to those skilled in the art. Thus, the invention in its broader aspects is therefore not limited to the specific details, representative apparatus and method, and illustrative examples shown and described. Accordingly, departures may be made from such details without departing from the spirit or scope of applicants' general inventive concept.

What is claimed is:

1. A hydrogel wound dressing having a wound-contact side and a non-wound-contact side, wherein a rate of moisture evaporation from said non-wound-contact side is faster than a rate of moisture evaporation from said wound-contact side.

2. The hydrogel wound dressing of claim 1, wherein the rate of moisture evaporation from said non-wound-contact side being faster than a rate of moisture evaporation from said wound-contact side causes the dressing to curl toward said non-wound-contact side upon drying.

3. The hydrogel wound dressing of claim 1 comprising disulfide-crosslinked keratin proteins wherein the rate of moisture evaporation from said non-wound-contact side is faster than the rate of moisture evaporation from said wound-contact side as a result of a manufacturing process comprising disulfide crosslinkage and gelation of a pre-hydrogel material while the pre-hydrogel material is coated on a surface and exposed to air.

4. The hydrogel wound dressing of claim 3, wherein said surface comprises an engraving with a mirror image of a message such that when the hydrogel is released from the surface, the engraving becomes a legible version of the message on the finished hydrogel.

5. The hydrogel wound dressing of claim 4, wherein said legible message is the word “UP”.

6. The hydrogel wound dressing of claim 3, comprising a layer of a non-keratin hydrogel affixed to the non-wound-contact side.

7. The hydrogel wound dressing of claim 6, wherein the non-keratin hydrogel comprises calcium alginate, CMC, and a plasticizer.

8. The hydrogel wound dressing of claim 7, wherein the plasticizer is selected from the group consisting of glycerin, propylene glycol, glyceryl polyacrylate, and triacetin.

9. The hydrogel wound dressing of claim 6, wherein the non-keratin hydrogel is cured on a surface comprising an engraving with a mirror image of a message such that when the non-keratin hydrogel is released from the surface, the engraving becomes a legible version of the message on the released non-keratin hydrogel.

10. The hydrogel wound dressing of claim 9, wherein said legible message is the word “UP” and the non-keratin hydrogel cured on the surface comprises a pigment.

11. The hydrogel wound dressing of claim 10, wherein said pigment is calcium stearate.

12. The hydrogel wound dressing of claim 1, wherein the wound-contact side and the non-wound-contact side comprise a non-keratin hydrogel and are bonded together to form a bilayer structure.

13. The hydrogel wound dressing of claim 12, wherein the non-keratin hydrogel of the non-wound-contact side comprises crosslinked gelatin and the non-keratin hydrogel of the wound-contact side comprises calcium alginate.

14. The hydrogel wound dressing of claim 13 further comprising a mesh.

15. The hydrogel wound dressing of claim 12, further comprising a therapeutic agent within a hydrogel layer.

16. The hydrogel wound dressing of claim 15 wherein said therapeutic agent is selected from the group consisting of polyhexamethylene biguanide hydrochloride, chlorhexidine gluconate, PVP-iodine, silver, and bismuth tribromophenate.

17. The hydrogel wound dressing of claim 6, further comprising a therapeutic agent within a hydrogel layer.

18. The wound dressing of claim 17 wherein said therapeutic agent is selected from the group consisting of polyhexamethylene biguanide hydrochloride, chlorhexidine gluconate, PVP-iodine, silver, and bismuth tribromophenate.

19. A process for making a hydrogel wound dressing, the process comprising:
adhering at least one layer of a human keratin matrix (“a first layer”) to at least one layer of either a substrate or a hydrogel (“a second layer”); and
compressing the first layer and the second layer until a desired thickness of the first layer and the second layer is obtained.

20. A process for manufacturing a composite comprising a human keratin matrix, said process comprising:
a) providing a sheet of the human keratin matrix;
b) providing a first aqueous solution comprising sodium alginate, CMC, and a plasticizer;
c) providing a second aqueous solution comprising a divalent or trivalent salt capable of crosslinking soluble alginate into an insoluble gel;
d) providing a surface comprising an engraving with a mirror image of a message that, upon releasing the insoluble gel from the surface, becomes a legible feature of the insoluble gel and filling the engraving with the first solution and a pigment;
e) coating the first solution over the surface;
f) placing the human keratin matrix over the surface coated with the first solution;
g) compressing the surface, the insoluble gel, and the human keratin matrix together until a desired thickness is achieved;
h) flooding the human keratin matrix with the second solution and allowing sufficient time for the second solution to permeate through the human keratin matrix and into the first solution coated over the surface to produce the composite; and
i) removing the composite from the surface and rinsing the composite with water to remove any excess second solution on the composite.

21. The process of claim 20, further comprising placing a mesh over the surface after coating the first solution over the surface and further coating the first solution over the mesh.

22. A wound dressing having a wound-contact side and a non-wound-contact side wherein the wound-contact side comprises keratin in a non-hydrogel state and the non-wound-contact side comprises a non-keratin hydrogel.

23. The wound dressing of claim 22, wherein said keratin comprises at least one layer of agglutinated particles.

24. The wound dressing of claim 22, wherein said non-keratin hydrogel comprises a mesh.

25. The wound dressing of claim 22, further comprising a therapeutic agent within a hydrogel layer.

26. The wound dressing of claim 25 wherein said therapeutic agent is selected from the group consisting of polyhexamethylene biguanide hydrochloride, chlorhexidine gluconate, PVP-iodine, silver, and bismuth tribromophenate.

27. A process of forming a human keratin matrix, the process comprising:
mixing an aqueous solution of keratin with a mater-miscible organic solvent to obtain keratin particles;
collecting the keratin particles;
re-suspending the keratin particles in water to produce a keratin slurry;
applying the keratin slurry to a substrate; and
drying the substrate with the keratin slurry so as to bond the keratin particles of the keratin slurry to the substrate.

28. A process of forming the wound dressing of claim 22, said process comprising:
a) mixing an aqueous solution of keratin with a water-miscible organic solvent to obtain keratin particles;
b) collecting the keratin particles and re-suspending the keratin particles in water to produce a slurry of keratin particles;
c) providing a substrate;
d) spreading the slurry of keratin particles on the substrate; and
e) drying the substrate to agglutinate the keratin particles and to bond the agglutinated keratin particles to the substrate.
